(19) United States
(12) Patent Application Publication
(10) Pub. No.: US 2005/0165115 A1
(43) Pub. Date: Jul. 28, 2005

(54) Methods and compositions for predicting compliance with an antidepressant treatment regimen

(76) Inventors: Greer M. Murphy, Stanford, CA (US); Alan F. Schatzberg, Los Altos, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/010,244

(22) Filed: Dec. 9, 2004

Related U.S. Application Data
(60) Provisional application No. 60/528,607, filed on Dec. 10, 2003.
(60) Provisional application No. 60/529,452, filed on Dec. 11, 2003.

(51) Int. Cl. A61K 31/137; C12Q 1/68

(52) U.S. Cl. 514/649; 435/6

(57) ABSTRACT

Methods and compositions are provided for predicting whether a subject will comply with an antidepressant treatment regimen. In practicing the subject methods, a subject's serotonin transporter gene-linked polymorphic region (5HTTLPR) is genotyped. Based on the identified genotype, a particular antidepressant treatment regimen suitable for the patient is determined. Also provided are kits for practicing the subject methods.

### CROSS-REFERENCE

This application claims the benefit of U.S. Provisional Application Nos. 60/528,607, filed Dec. 10, 2003, and 60/529,452, filed Dec. 11, 2003, which applications are incorporated herein by reference in their entirety.

### GOVERNMENT RIGHTS

This invention was made with government support from the Department of Veterans Affairs. The United States Government may have certain rights in this invention.

### INTRODUCTION

### BACKGROUND OF THE INVENTION

Depression is a difficult mental disorder to treat. Patients having such a disorder are often reluctant to seek the medical attention necessary to diagnose the disorder. Such reluctance is often related to the patient's fear of the stigma associated with seeking psychiatric help or to the patient's feeling of worthlessness associated with depression. Moreover, once the patient seeks competent psychiatric help, it is difficult to successfully treat the disorder through psychotherapeutic approaches alone.

In an effort to treat depression, a variety of antidepressant pharmacological agents have been developed. Among these are the selective serotonin reuptake inhibitors (SSRI), such as setraline (registered trademark ZOLOFT™-Pfizer), fluoxetine (registered trademark PROZAC™-Eli Lilly), paroxetine (trade name PAXIL™-Smith Kline Beecham), and fluvoxamine (trade name LUVOX™) and Citalopram (trade name CELEXA™). Other examples of antidepressant compositions include: tricyclic antidepressants, such as those sold under the registered trademark ELAVIL™ (Merck, Sharpe and Dohme); α2-adrenergic receptor antagonists, such as mirtazapine; aminoketone antidepressants such as bupropion; and lithium, a metal used to treat bipolar disorder.

While these pharmacological agents are efficacious, they are also potent, often generating problematic side effects such as lethargy, clouded thinking, a lack of ability to concentrate, and sexual dysfunction. In many cases, the side effects of these drugs become so severe that the patient discontinues the treatment regimen. Furthermore, these drugs often take about six to eight weeks to exhibit any desirable therapeutic effects. Accordingly, this time period can be prolonged when the correct drug or combinations of drugs has to be determined, by trial and error, before any therapeutic effects are observed. Furthermore, current research is beginning to unveil that many of these drugs produce undesirable physiological side effects (Sipgset, O. Drug Saf. 1999. 20(3):277-287; Pache, P. Curr. Med Chem. 1999. 6(6): 46-480) such as drowsiness, low blood pressure, nausea, headache, restlessness, anxiety, diarrhea/loose stool, and sexual dysfunction.

In view of such severe side effects, patients being administered an antidepressant for the treatment of depression may not comply with the treatment regimen. Oftentimes, such non-compliance with the treatment regimen includes the patient unilaterally decreasing the prescribed dosage of the antidepressant in order to decrease the experienced side-effects. However, such actions only exacerbate the underlying mental condition that the treatment regimen is aimed at treating. Additionally, such non-compliance with the treatment regimen also includes discontinuation of the treatment regimen by the patient. Although the clinician may switch the patient to another antidepressant agent hoping for a better response and compliance, there is no rational means for determining the best choice. Frequent switching of medications increases patient suffering and wastes health care dollars.

Therefore, there is a need for the development of a new method for predicting a patient's compliance with an antidepressant treatment regimen in order to select the optimal antidepressant treatment regimen for the patient. The present invention satisfies this need.

### RELEVANT LITERATURE

References of interest include: Perlis et al., Biol. Psychiatry 54:879-883 (2003); Kim et al., Neuroreport 11 (1):215-219 (2000); Yoshid et al., Prog Neuropsychopharmacol Biol Psychiatry. 26(2):383-386 (2002); Schatzberg et al., Am J Geriatr Psychiatry 10(5):541-550 (2002); Murphy et al., Am J Psychiatry 160(10):1830-1835 (2003); Edenberg et al., Psychiatr Genet 8(3):193-195 (1988); Heils et al., J. Neural Transm. 102:247 (1995); Heils et al., J. Neurochem 66:2621 (1996), Lesch et al., Science 274:1527 (1996); Furlong et al., Am J Med Genet 81(1):58-63 (1998); Menza et al., J Geriatr Psychiatry Neurol 12(2):49-52 (1999); and Rosenthal et al., Mol Psychiatry 3(2):175-7 (1998).

### SUMMARY OF THE INVENTION

Methods and compositions are provided for predicting whether a subject will comply with an antidepressant treatment regimen. In practicing the subject methods, a subject's serotonin transporter gene-linked polymorphic region (5HTTLPR) is genotyped. Based on the identified genotype, a particular antidepressant treatment regimen suitable for the patient is determined. Also provided are kits for practicing the subject methods.

Aspects of the invention include methods for predicting or evaluating whether a subject will comply with an antidepressant treatment regimen by first determining the subject's serotonin transporter gene-linked polymorphic region (5HTTLPR) genotype and then using the determined genotype to predict whether the subject will comply with an antidepressant treatment regimen. In such methods the subject may be a mammal, such as a human. In addition, the subject of such methods may possess a disorder, such as Major Depressive Disorder (MDD) or any other disorder that may be treated with an antidepressant treatment regimen.

Furthermore, in such methods, one form of the 5HTTLPR genotype may indicate a decreased compliance to a particular antidepressant treatment regimen, as compared to other forms of the 5HTTLPR genotype. The identified 5HTTLPR genotype may be Long/Long (L/L), Long/Short (L/S), or Short/Short (S/S). In some embodiments, the L/L genotype may be associated with an increased compliance with a serotonin selective reuptake inhibitor (SSRI) antidepressant treatment regimen, e.g., where the SSRI is paroxetine. In some embodiments, the S/S genotype may be associated with an increased compliance with a noradrenergic and specific serotonergic antidepressant treatment regimen, e.g., where the noradrenergic and specific serotonergic antidepressant is mirtazapine.

Also provided are kits that include reagents for determining a subject's 5HTTLPR genotype, and instructions for using the subject's genotype for evaluating whether the subject will comply with a SSRI treatment regimen.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a table listing the baseline characteristics and clinical course for the patients treated with paroxetine and mirtazapine stratified by 5HTTLPR genotype. Abbreviations used in the table include: GDS, Geriatric Depression Scale; HDRS-17 (also referred to as HAMD-17), Hamilton Depression Rating Scale; MMSE, Mini-Mental State Examination. Data is presented as mean (SD) unless otherwise indicated. The sample size was 244.

FIGS. 2A-D are graphs showing the HAMD-17 and GDS scores of patients administered paroxetine and mitrazapine stratified by 5HTTLPR genotype (S/S, S/L, L/L).

FIGS. 3A-B are graphs showing the effect of 5HTTLPR polymorphism on discontinuation of treatment regimen by patients administered either paroxetine or mitrazapine.

### DETAILED DESCRIPTION OF THE INVENTION

Methods and compositions are provided for determining whether a subject will comply with an antidepressant treatment regimen. In practicing the subject methods, a subject's serotonin transporter gene-linked polymorphic region (5HTTLPR) is genotyped. Based on the identified genotype the subject possesses, a particular antidepressant treatment regimen suitable for the patient is determined. Also provided are kits for practicing the subject methods.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “an antidepressant” includes a plurality of such antidepressants and reference to “the antidepressant ” includes reference to one or more antidepressant and equivalents thereof known to those skilled in the art, and so forth.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In further describing the subject invention, the methods will be described first, followed by a review of the representative applications in with the methods find use, and kits thereof that find use in practicing the subject methods.

Methods

As summarized above, the subject invention provides methods for evaluating, i.e., determining or predicting, whether a subject will comply with a particular antidepressant treatment regimen. In the subject methods, one first determines the subject's serotonin transporter gene-linked polymorphic region genotype. The resultant determined genotype is then used to predict whether the subject will comply with a particular antidepressant treatment.

By “antidepressant treatment regimen” is meant a systematic regulated course of therapy pursued with a view to improve a subject's health, where the course of therapy includes administration of at least one antidepressant pharmacological agent. In a given antidepressant treatment regimen, an antidepressant pharmacological agent is administered to a patient at least once over a given period of time, where administration may be pursuant to a dosage schedule of times, e.g., daily, weekly, biweekly, monthly etc, and amount of agent.

By “comply” is meant to act in accordance with the treatment regimen. In other words, to follow a prescribed antidepressant treatment regimen. As such, a subject complies with a given antidepressant treatment regimen if the subject follows the pharmacological agent dosing schedule of the treatment regimen, so that the pharmacological agent is only taken at times and in amounts as defined by the dosing schedule.

As summarized above, one first determines the subject's serotonin transporter gene-linked polymorphic region genotype. The human serotonin transporter (5HTT) is encoded by a single gene (SLC6A4) found on chromosome 17q12 (Ramamoorthy et al., Proc. Natl. Acad. Sci. USA 90:2542 (1993); Gelernter et al., Hum. Genet. 95:677 (1995). The serotonin transporter regulates the magnitude and duration of serotonergic responses. An insertion/deletion polymorphism consisting of a 44 base pair segment in the transcriptional control region 5′ upstream to the 5HTT coding sequence has previously been identified and is termed as either the Long form (insertion) or the short form (deletion) of the serotonin transporter gene-linked polymorphic region (5HTTLPR). Various forms of the 5HTTLPR include Long/Long (L/L), Long/Short (L/S), Short/Short (S/S). Accordingly, genotypes of interest include L/L, L/S and S/S.

As noted above, the subject invention provides methods for predicting whether a subject will comply with an antidepressant treatment regimen based on the subject's determined 5HTLPR genotype. As used herein, “genotyping” a subject (or DNA sample) for a polymorphic allele at a defined genomic locus or “determining the genotype” at a polymorphic allelic site, means detecting or identifying which forms of the allele are present in a subject (or a sample). As is well known in the art, an individual may be heterozygous or homozygous for a particular allele.

As used herein, the process of detecting an allele or polymorphism includes any suitable method as is known in the art. Accordingly, an allele or a polymorphism may be detected directly or it may be detected indirectly. Indirect detection of an allele or polymorphism includes the detection of an allele or polymorphism that is in linkage disequilibrium with a functional polymorphism/allele. “Linkage disequilibrium” as used herein refers to the tendency of specific alleles at different genomic locations to occur together more frequently than would be expected by chance. Polymorphisms/alleles are evidenced in the genomic DNA of a subject, but may also be detectable from RNA, cDNA or protein sequences transcribed or translated from this region, as will be apparent to one skilled in the art.

Alternatively, polymorphic alleles may be directly detected by determining the DNA polynucleotide sequence, or by detecting the corresponding sequence in RNA transcripts from the polymorphic gene, or where the nucleic acid polymorphism results in a change in an encoded protein by detecting such amino acid sequence changes in encoded proteins; using any suitable technique as is known in the art. Polynucleotides utilized for genotyping are typically genomic DNA, or a polynucleotide fragment derived from a genomic polynucleotide sequence. The polymorphism may be detected in a method that comprises contacting a polynucleotide or protein sample from an individual with a specific binding agent for the polymorphism and determining whether the agent binds to the polynucleotide or protein, where the binding indicates that the polymorphism is present. The binding agent may also bind to flanking nucleotides and amino acids on one or both sides of the polymorphism, for example at least 2, 5, 10, 15 or more flanking nucleotide or amino acids in total or on each side.

The binding agent may be a polynucleotide (single or double stranded) typically with a length of at least 10 nucleotides, for example at least 15, 20, 30, or more nucleotides. A polynucleotide agent which is used in the method will generally bind to the polymorphism of interest, and the flanking sequence, in a sequence specific manner (e.g. hybridize in accordance with Watson-Crick base pairing) and thus typically has a sequence which is fully or partially complementary to the sequence of the polymorphism and flanking region. The binding agent may be a molecule that is structurally similar to polynucleotides that comprises units (such as purine or pyrimidine analogs, peptide nucleic acids, or RNA derivatives) able to participate in Watson-Crick base pairing. The agent may be a protein, typically with a length of at least 10 amino acids, such as at least 20, 30, 50, or 100 or more amino acids. The agent may be an antibody (including a fragment of such an antibody that is capable of binding the polymorphism.

In one embodiment the polynucleotide agent is able to act as a primer for a PCR reaction where two agents are able to bind on either side of the deletion/insertion region of the 5HTTLPR. Thus, the PCR products that will be produced will be of two different sizes depending on the alleles present in the subject. The products can then be assayed on gel electrophoresis to analyze the length of the products and determine the genotype of the subject.

In another embodiment the polynucleotide agent is able to act as a primer for a PCR reaction only if it binds a polynucleotide containing the polymorphism (i.e. a sequence- or allele-specific PCR system). Thus a PCR product will only be produced if the polymorphism is present in the polynucleotide of the individual, and the presence of the polymorphism is determined by the detection of the PCR product. Preferably the region of the primer which is complementary to the polymorphism is at or near the 3′ end the primer. In one embodiment of this system the polynucleotide the agent will bind to the wild-type sequence but will not act as a primer for a PCR reaction.

In another embodiment, the method may be a Restriction Fragment Length Polymorphism (RFLP) based system. This can be used if the presence of the polymorphism in the polynucleotide creates or destroys a restriction site that is recognized by a restriction enzyme. Thus treatment of a polynucleotide that has such a polymorphism will lead to different products being produced compared to the corresponding wild-type sequence. Thus the detection of the presence of particular restriction digest products can be used to determine the presence of the polymorphism.

In yet another embodiment, the method may be an oligonucleotide microarray based system. In such an embodiment, the binding agents may be Single Nucleotide Polymorphisms (SNPs) representing a major source of genetic variation which may be used to screen genomic DNA from the subject to determine their genotype.

Various other detection techniques suitable for use in the present methods will be apparent to those conversant with methods of detecting, identifying, and/or distinguishing polymorphisms. Such detection techniques include but are not limited to direct sequencing, use of “molecular beacons” (oligonucleotide probes that fluoresce upon hybridization, useful in real-time fluorescence PCR; see e.g., Marras et al., Genet Anal 14:151 (1999)); electrochemical detection (reduction or oxidation of DNA bases or sugars; see U.S. Pat. No. 5,871,918 to Thorp et al.); rolling circle amplification (see, e.g., Gusev et al., Am J Pathol 159:63 (2001)); Third Wave Technologies (Madison Wis.) INVADER non-PCR based detection method (see, e.g., Lieder, Advance for Laboratory Managers, 70 (2000)).

Accordingly, any suitable detection technique as is known in the art may be utilized in the present methods to genotype the subject. Furthermore, suitable biological specimens to use for genotyping the subject are those which comprise cells and DNA and include, but are not limited to blood or blood components, dried blood spots, urine, buccal swabs and saliva.

In practicing the subject methods, once the genotype of the subject is determined, it is used to predict whether the subject will be compliant with an antidepressant treatment regimen and will not discontinue the treatment. The antidepressant agents of the particular antidepressant treatment region may be administered daily, more then once daily etc., as described above. The treatment regimen may be for three months, to six months, or twelve months, or a period of time determined suitable for treatment by a physician. The dosage and frequency of administration of the antidepressant during the treatment regimen may be changed by the physician for a variety of reasons, such as pregnancy, development of allergic reaction, or ineffectiveness of the specific antidepressant.

Based on the subject's 5HTTLPR genotype, a prediction may be made whether the subject will comply with a particular antidepressant treatment regimen compared to other antidepressant treatment regimens, e.g., regimens in which other pharmacological agents are employed. Therefore, the selected antidepressant treatment regimen is more effective and rationally based.

In some embodiments, where the subject possesses a 5HTTLPR L/L genotype, a prediction may be made that the subject will be more compliant with an antidepressant treatment regimen that involves an SSRI, as compared to other subjects that possess other forms of the 5HTTLPR genotype. Accordingly, such a prediction may be used to administer the subject a specific appropriate antidepressant where the subject will comply with the treatment regimen, as compared to other specific antidepressants. A specific antidepressant treatment regimen that a subject possessing a 5HTTLPR L/L genotype will be more compliant with is the particular SSRI known as paroxetine.

In other embodiments, where the subject possesses a 5HTTLPR S/S genotype a prediction may be made that the subject will be more compliant with a antidepressant treatment regimen that involves a noradrenergic and specific serotonergic antidepressant, as compared to other subjects that possess other forms of the 5HTTLPR genotype. Accordingly, such a prediction may be used to administer the subject a specific appropriate antidepressant where the subject will comply with the treatment regimen, as compared to other specific antidepressants. A specific antidepressant treatment regimen that a subject possessing a 5HTTLPR S/S genotype will be more compliant with is one in which the 2-adrenergic receptor antagonist mirtazapine is administered.

In other embodiments, where the subject possesses a 5HTTLPR L/L genotype, a prediction may be made that the subject will not discontinue use of an antidepressant treatment regimen that involves an SSRI, as compared to other subjects that possess other forms of the 5HTTLPR genotype. Accordingly, such a prediction may be used to administer the subject a specific appropriate antidepressant where the subject will not discontinue the treatment regimen, as compared to other specific antidepressants. A specific antidepressant treatment regimen that a subject possessing a 5HTTLPR L/L genotype will not discontinue use is the particular SSRI known as paroxetine.

In other embodiments, where the subject possesses a 5HTTLPR S/S genotype a prediction may be made that the subject will not discontinue use of an antidepressant treatment regimen that involves a noradrenergic and specific serotonergic antidepressant, as compared to other subjects that possess other forms of the 5HTTLPR genotype. Accordingly, such a prediction may be used to administer the subject a specific appropriate antidepressant where the subject will not discontinue the treatment regimen, as compared to other specific antidepressants. A specific antidepressant treatment regimen that a subject possessing a 5HTTLPR S/S genotype will not discontinue use is the particular noradrenergic and specific serotonergic antidepressant mirtazapine.

Classes of antidepressant compounds for which compliance may be predicted according to the subject methods include, but are not limited to, SSRIs, noradrenergic and specific serotonergic antidepressant, Heterocyclic antidepressants, and Monoamine oxidase inhibitors (MAOIs). Examples of SSRI agents include Fluoxetine (PROZAC™); Fluvoxamine (LUVOX™); Paroxetine (PAXIL™); Sertraline (ZOLOFT™); Citalopram (CELEXA™); etc., including duel norepinephrine/serotonin uptake blockers, e.g., venlafaxine (EFFEXOR™), duloxetine, milnacipran, etc. Also of interest are serotonin 1-a partial agonists, e.g., gepirone, buspirone, etc. SSRIs directly interact with the serotonin transporter to increase the availability of serotonin in the synaptic cleft. In certain embodiments, however, the methods are not employed to predict compliance with a treatment regimen that employs Fluoxetine (PROZAC™). Alternatively, noradrenergic and specific serotonergic antidepressants, such as Mitazapine (REMERON™) induce the release of norepinephirine as well as serotonin in the brain through antagonism of the α-2-adrenergic receptors on noradrenergic and serotonergic neurons. Heterocyclic antidepressants, such as tricyclic (the tertiary amines amitriptyline and imipramine and their secondary amine metabolites nortriptyline and desipramine), modified tricyclic, and tetracyclic antidepressants, primarily increase the availability of norepinephrine and, to some extent, of serotonin by blocking reuptake in the synaptic cleft. MAOIs inhibit the oxidative deamination of the three classes of biogenic amines, such as norepinephrine, dopamine, and serotonin, and other phenylethylamines.

Utility

The subject methods find use in a variety of different applications where it is desirable to make rational medication treatment choices before a treatment regimen is initiated. Antidepressants are potent pharmaceuticals prescribed for depression. Such depression states are those defined in the Diagnostic and Statistical Manual of Mental Disorders, third edition (DSM III), American Psychiatric Association, Washington, D.C. (1980), (DSM III, 296.2× to 296.6× and 301.13), including that characterized by anxiety or obsessional neuroses (DSM III, 300.40), or atypical depression (DSM III, 296.70 and 296.82), e.g., accompanied by a personality disorder.

However, oftentimes antidepressants are accompanied by severe side effects. In many cases the side effects of these drugs become so severe that the subject discontinues the treatment regimen. Therefore, it is beneficial to select the appropriate antidepressant that results in the subject's compliance with the treatment regimen. The subject methods find use in such applications where a particular antidepressant treatment regimen suitable for the patient is determined based on the identified genotype the subject possesses.

A variety of subjects are treatable according to the subject methods. Generally such hosts are “mammals” or “mammalian,” where these terms are used broadly to describe organisms which are within the class mammalian. In many embodiments the subjects of the present methods will be humans.

In some embodiments, the subject methods include first diagnosing a subject for a depressive state and then determining the subject's serotonin transporter gene-linked polymorphic region (5HTTLPR) genotype. Based on the subject's 5HTTLPR genotype, determining a particular antidepressant treatment regimen suitable for the subject as compared to other antidepressant treatment regimens, e.g., regimens in which other pharmacological agents are employed, and administering the antidepressant treatment regimen to the subject for treatment of the depressive state.

In such embodiments, depression is diagnosed by a health care provider or by referral to a psychiatrist, psychologist, or specially trained mental health professional, which health professional may administer a psychological exam. Suitable psychological exams include, but are not limited to, special screening test for depression, such as the Beck Depression Inventory or the Hamilton Rating Scale.

In some embodiments, where the subject possesses a 5HTTLPR L/L genotype, a prediction may be made that the subject will be more compliant with an antidepressant treatment regimen (e.g., will not discontinue use) that involves an SSRI, as compared to other subjects that possess other forms of the 5HTTLPR genotype. A specific antidepressant treatment regimen that a subject possessing a 5HTTLPR L/L genotype will be more compliant with (e.g., will not discontinue use) is the particular SSRI known as paroxetine.

In other embodiments, where the subject possesses a 5HTTLPR S/S genotype a prediction may be made that the subject will be more compliant with a antidepressant treatment regimen (e.g., will not discontinue use) that involves a noradrenergic and specific serotonergic antidepressant, as compared to other subjects that possess other forms of the 5HTTLPR genotype. A specific antidepressant treatment regimen that a subject possessing a 5HTTLPR S/S genotype will not discontinue use (e.g., will not discontinue use) is the particular noradrenergic and specific serotonergic antidepressant mirtazapine.

Kits

Also provided are kits that find use in practicing the subject methods, as described above. For example, in some embodiments, kits for practicing the subject methods may include reagents for determining a subject's serotonin transporter gene-linked polymorphic region (5HTTLPR) genotype. Such reagents may include buffers, solutions, enzymes, and PCR primers useful for determining a subject's 5HTTLPR genotype. In some embodiments, the kits may also include binding agents, such as polynucleotides (e.g., primer), e.g. for use in detecting the polymorphic alleles, for example, by PCR. In other embodiments, the kits may include an oligonucleotide array for use in detecting the polymorphic alleles.

In addition to the above components, the subject kits may further include instructions for predicting or evaluating whether the subject will comply with a SSRI treatment regimen. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

I. Material and Methods

A. Study Design

The study group included 255 outpatients 65 years of age or older with major depressive disorder (MDD) who were randomized to mirtazapine or paroxetine in a double-blind design at 18 U.S. centers. Details of the study design and overall clinical outcomes have been previously described (Schatzberg et al., Am J Geriatr Psychiatry 10(5):541-550 (2002)). Of the 254 evaluable patients, 246 received study medication (mirtazapine N=124 or paroxetine N=122) and at least one post-baseline assessment. At baseline all patients scored above the age-adjusted 25th percentile on the Mini Mental State Exam (MMSE), and all had Hamilton Depression Rating Scale (HDRS-17) scores ≧18. Starting doses were 15 mg for mirtazapine and 20 mg for paroxetine. Dosage for both medications was increased to 30 mg after 14 days, with the option to increase to 45 mg of mirtazapine or 40 mg of paroxetine on subsequent visits. Exclusion criteria were clinically significant unstable medical illness, history of treatment resistance, principal psychiatric diagnoses other than MDD, use of other psychotropics, and electroconvulsive therapy (ECT) within the previous 6 months.

B. Genotyping

Genomic DNA was extracted from EDTA-treated frozen whole blood by using a Puregene Kit (Gentra Systems, Minneapolis). 5HTTLPR genotypes were determined by PCR and gel electrophoresis as described in Edenberg et al., Psychiatr. Genet. 8(3):193-195 (1998).

C. Analysis

Plasma drug levels were obtained after 4 weeks of treatment as described (Murphy et al., Biol Psychiatry 54:665-673 (2003)). Efficacy outcome measures (determined at baseline and at day 7,14, 21, 28, 42, and 56) included the Hamilton Depression Rating Scale (HDRS-17) (Hamilton et al., Br J Soc Clin Psychol. 6(4):278-296 (1967)) and the Geriatric Depression Scale (GDS) (Yesavage et al., J. Psychiat. Res. 17:37-49 (1983)). Measures related to adverse events included Medication Compliance, Severity of Adverse Events, Time to Discontinuation of Treatment Regimen due to an adverse event, and number of discontinuations due to an adverse event. Statistical methods included analyses of covariance (adjusted for baseline scores), Cochran-Mantel-Haenszel statistics, and Kaplan-Meier survival analyses. To test for interactions between the 5HTTLPR and the serotonin 2A receptor (5HT2A) 102 T/C variant that we previously found to affect paroxetine discontinuations (Murphy et al., 2003), we used a Cox proportional hazard regression analysis with an interaction term. Analyses were performed for the entire group and also with 20 ethnic minority patients omitted.

II. Results

A. Overview

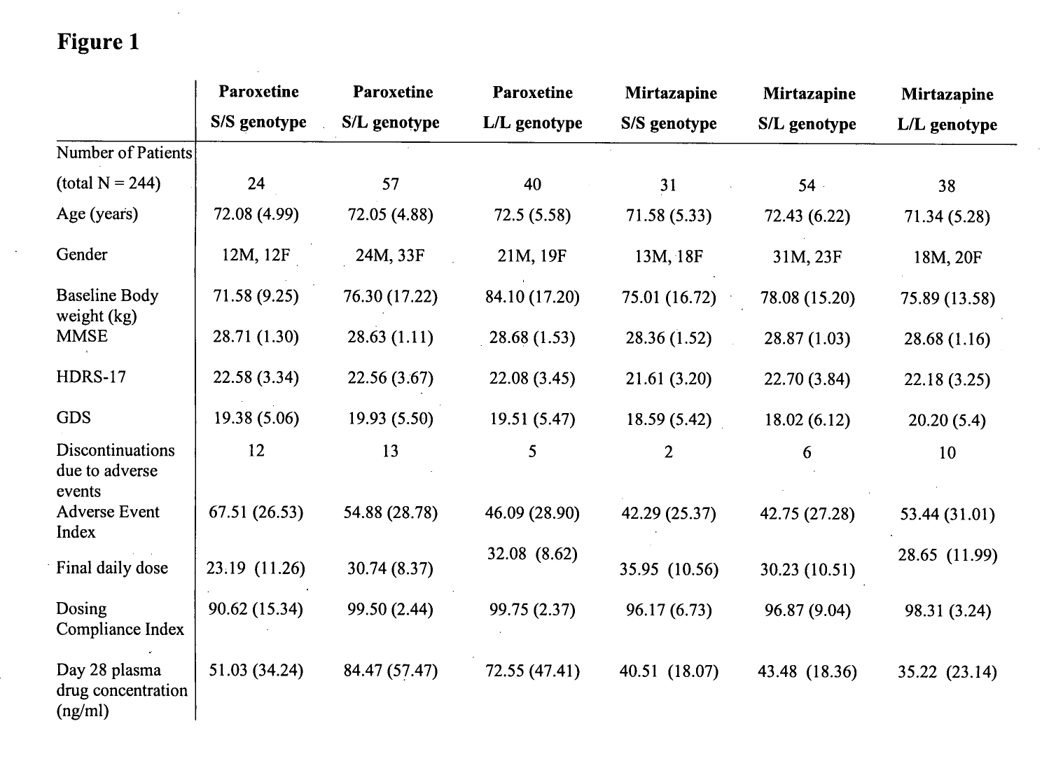

Baseline characteristics and clinical course for patients treated with paroxetine and mirtazapine stratified by 5HTTLPR genotype are presented in FIG. 1. Overall allele frequencies for the entire sample were: L=0.547, S=0.453. Overall genotype frequencies for the entire sample were: 32.0% L/L, 45.5% S/L, 22.5% S/S. There were no significant differences between the paroxetine and mirtazapine groups in allele or genotype frequencies. For the full sample and for the treatment sub-samples there were no deviations from Hardy-Weinberg equilibrium. There were no differences among the genotype groups in mean age, numbers of males and females, numbers of ethnic minorities, or baseline MMSE for either treatment group. For the paroxetine group, baseline body weight was significantly greater for the L/L genotype group than for the S/L (p<0.016) and S/S groups (p<0.002). Because plasma drug concentrations and pharmacokinetics can potentially be affected by body weight, we included baseline body weight as a covariate in all subsequent analyses. There were no significant differences in baseline body weight among the genotype groups for the mirtazapine-treated subjects. There were two subjects lacking baseline body weight measurements, and these subjects were excluded from subsequent analyses, giving a final N of subjects of 244.

B. Efficacy

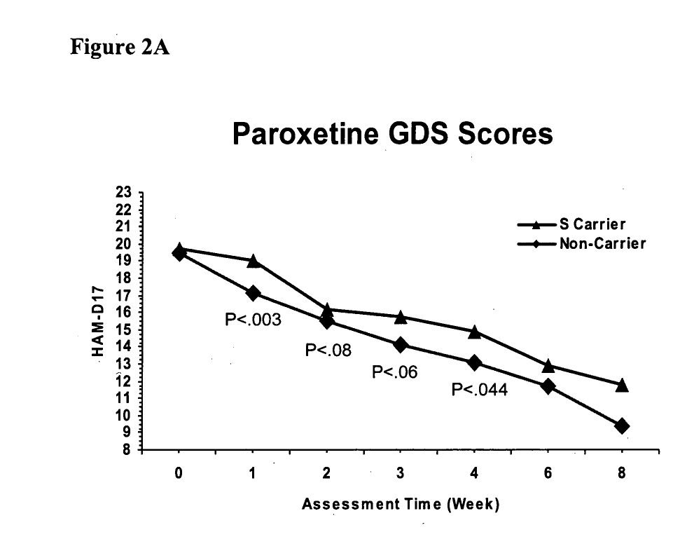

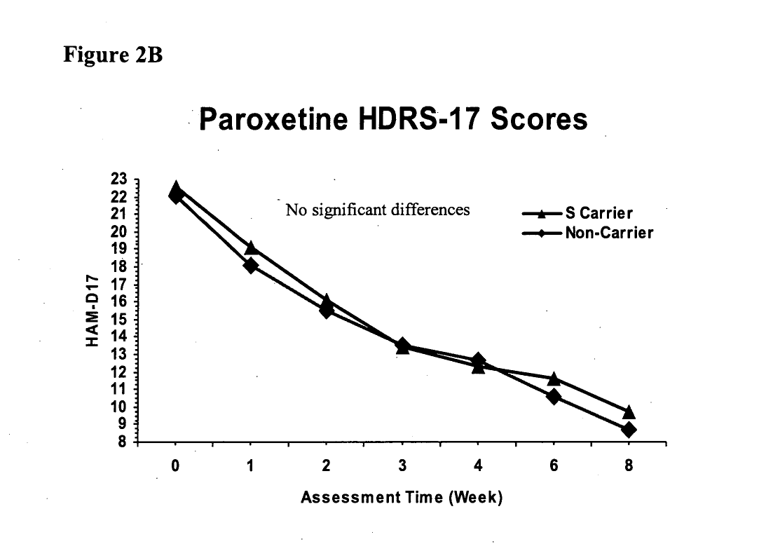

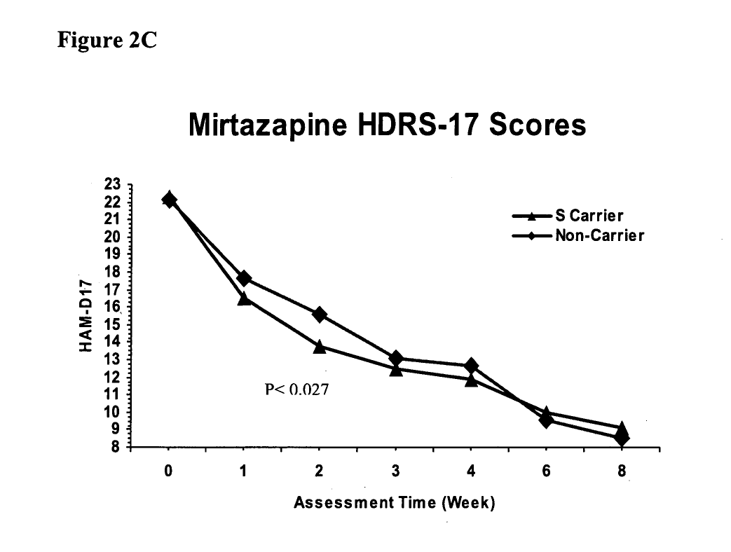

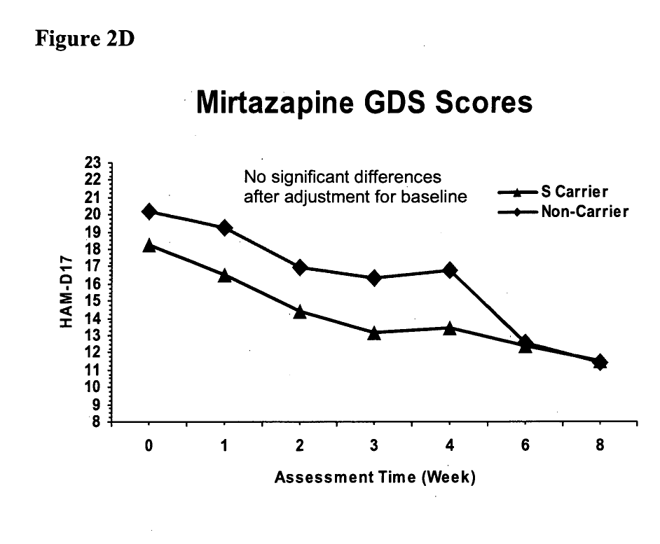

For both paroxetine and mirtazapine, when patients were stratified by 5HTTLPR genotype (S/S, S/L, and L/L), there were no significant differences among the three genotype groups at any time point for either the HAMD-17 or the GDS. Prior studies had combined S/S and S/L genotypes into an “S carrier” group for efficacy analysis (Smeraldi et al., Mol Psychiatry 3(6):508-511 (1998); Pollock et al., Neuropsychopharmacology 23(5):587-590 (2000)). For paroxetine treated patients, carriers of the S allele showed significantly higher scores on the GDS (more depressed) at days 7 (p=0.003) and 28 (p=0.04), after adjusting for baseline body weight (FIG. 2A). No differences were seen on the HDRS (FIG. 2B). S allele carriers treated with mirtazapine showed lower HDRS scores at day 14 (less depressed; p<0.27; FIG. 2C). GDS scores for mirtazapine-treated patients with the S/S and S/L genotypes were significantly lower at baseline than were those for patients with the L/L genotype (p<0.26; FIG. 2D). After adjusting for this difference, GDS scores for mirtazapine-treated S carriers were not significantly different from others at any subsequent time point.

C. Adverse Events

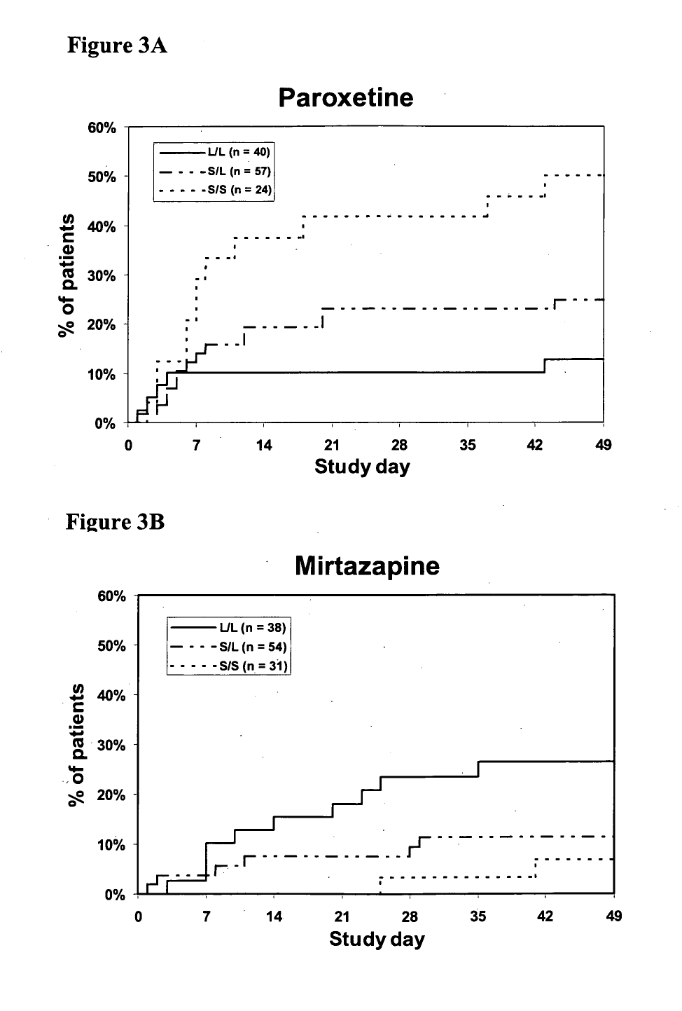

The 5HTTLPR polymorphism had a significant effect on discontinuations of the treatment regimen due to adverse events for both medications. For paroxetine-treated patients, survival analyses showed that there was a positive linear relationship between the number of S alleles and probability of discontinuation due to adverse events at days 14, 21, 28, 42, and 49 (FIG. 3A; log rank tests, p values <0.05 for all). All statistical tests were adjusted for differences in baseline body weight. At the same time points, patients with the S/L genotype showed a significantly greater risk of discontinuation of the treatment regimen due to adverse events than did those with the L/L genotype, and patients with the S/S genotype also showed a greater severity of adverse events than those with the L/L genotype (p<0.02). Paroxetine-treated patients with the S/S genotype showed a lower final daily dose (p<0.001 for S/S vs. S/L and S/S vs. L/L), decreased dosing compliance (p<0.001 for S/S vs. S/L, and S/S vs. L/L), and lower plasma levels at day 28 (S/S vs. S/L, and S/S vs. L/L; p<0.05). Adverse events associated with S/S discontinuations included gastrointestinal complaints, fatigue, agitation, sweating, and dizziness.

Surprisingly, among mirtazapine-treated patients, the L allele was strongly associated with discontinuations due to adverse events. Survival analyses showed a positive linear relationship between the number of L alleles and probability of discontinuation due to adverse events at days 14, 21, 28, 42, and 49 (FIG. 3B; log rank tests, p values 0.05 to 0.009). The L/L genotype was also associated with a greater severity of adverse events (L/L vs. S/S; p=0.024), and a lower final daily dose (L/L vs. S/S, p=0.008). Among mirtazapine-treated patients, there were no significant differences in dosing compliance among the genotype groups, and no significant differences in plasma levels at day 28. Adverse events associated with L/L genotype discontinuations among mirtazapine-treated patients included drowsiness, dizziness, and anxiety.

It has been previously shown that the 5HT2A 102 T/C polymorphism is involved in predicting discontinuation of treatment regimen due to adverse events for paroxetine-treated patients (Murphy et al., Am J Psychiatry 160(10):1830-1835 (2003)). However, chi-squared analyses showed no significant associations between 5HT2A 102 T/C genotype and 5HTTLPR genotype for either drug (mirtazapine χ2=1.0, d.f.=4, NS; paroxetine χ2=7.1, d.f.=4, NS). Cox hazards regression analysis for paroxetine treated patients showed significant effects for 5HTTLPR and 5HT2A 102 T/C on discontinuation of treatment regimen due to adverse events, but the interaction term in the analysis was not significant. This finding indicates that 5HTTLPR and 5HT2A 102 T/C variants have additive, but not interactive effects on paroxetine discontinuations.

Analyses performed with the 20 ethnic minority patients removed from the sample gave similar results. For paroxetine-treated Caucasian patients, survival analyses showed a positive linear relationship between the number of S alleles and discontinuations due to adverse events at days 14, 21, 28, 42, and 49 (log rank tests; p values 0.035 to 0.008). Similarly, for mirtazapine-treated Caucasian patients, there was a positive linear relationship between the number of L alleles and discontinuation of treatment regimen due to adverse events at days 14, 21, 28, 42, and 49 (log rank tests; p<0.05 for all).

D. Analysis

These results show that the 5HTTLPR polymorphism is a marker for discontinuation of treatment regimen due to adverse events in geriatric patients treated with antidepressant agents, as exemplified by paroxetine and mirtazapine. For patients treated with paroxetine, discontinuation of treatment regimen was most frequent among those with the S/S genotype. The S/L genotype was associated with an intermediate frequency of discontinuation of treatment regimen, whereas L/L carriers had the fewest discontinuation of treatment regimen, indicating a gene dosage effect. Those with the S/S genotype also had a greater severity of adverse events. These effects were significant even after adjustment for differences in baseline body weight. Paroxetine-treated patients with the S/S genotype also showed lower medication compliance, resulting in lower plasma drug concentrations at day 28. In contrast, for patients treated with mirtazapine, discontinuation of treatment regimen was most frequent among those with the L/L genotype.

It has previously been demonstrated that the C/C genotype at the 5HT2A 102 T/C polymorphism is also a strong risk factor for discontinuation of treatment regimen due to adverse events among paroxetine treated patients (Murphy et al. 2003). Subjects with the 5HTTLPR S/S genotype and the 5HT2A 102 C/C genotype appear to be at exceptionally high risk for discontinuation of treatment regimen. Thus, whereas 7 of 10 paroxetine-treated subjects with the 5HTTLPR S/S and the 5HT2A C/C genotypes discontinued their treatment regimen due to adverse events, none of the 5 subjects with the L/L and T/T genotypes receiving paroxetine discontinued their treatment regimen. However, a Cox proportional hazards analysis showed that the increase in risk for discontinuation of treatment regimen due to S/S and C/C genotypes was additive, and not interactive. Therefore, the two effects are independent in this sample.

In summary, the above results show that the S allele at the 5HTTLPR polymorphism associated with adverse events in geriatric patients with major depression treated with paroxetine. This effect was independent of the previously described association of the 5HT2A 102 T/C polymorphism with paroxetine adverse events (Murphy at a., 2003). In addition, there was a modest effect of the 5HTTLPR polymorphism on paroxetine efficacy in this patient population. The 5HTTLPR is also important in determining discontinuation of treatment regimen due to adverse events during mirtazapine therapy, but surprisingly, the L allele confers greater risk with mirtazapine, rather than the S variant associated with discontinuation of treatment regimen among paroxetine-treated patients. Accordingly, differences in the mechanisms of action of mirtazapine and paroxetine account for the divergent effects of the 5HTTLPR on treatment outcome with these agents.

It is evident from the above description and results that the subject invention provides important new ways of predicting whether a subject will comply with a given antidepressant treatment regimen, even prior to start of the regimen. As such, the subject invention allows one to pick an appropriate treatment regimen for a given subject prior to starting the regimen, thereby reducing the problems associated with determination of an appropriate regimen by trial and error, where such problems include cost, adverse effects experience by the patient, including frustration etc., and the like. Accordingly, the present invention represents a significant contribution to the art.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of predicting whether a subject will comply with an antidepressant treatment regimen, said method comprising:
determining said subject's serotonin transporter gene-linked polymorphic region (5HTTLPR) genotype;
using said determined genotype to predict whether said subject will comply with said antidepressant treatment regimen.

2. The method according to claim 1, wherein said antidepressant treatment regimen comprises administration of an antidepressant agent to said subject.

3. The method according to claim 1, wherein said subject is a human.

4. The method according to claim 3, wherein said human subject possesses major depressive disorder (MDD).

5. The method according to claim 2, wherein said antidepressant agent is a serotonin selective reuptake inhibitor (SSRI) so that said antidepressant treatment regimen is a SSRI treatment regimen.

6. The method according to claim 5, wherein a 5HTTLPR Long/Long (L/L) genotype indicates an increased compliance with a SSRI treatment regimen.

7. The method according to claim 5, wherein said SSRI is paroxetine.

8. The method according to claim 2, where said antidepressant agent is a noradrenergic and specific serotonergic antidepressant so that said antidepressant treatment regimen is a noradrenergic and specific serotonergic antidepressant treatment regimen.

9. The method according to claim 8, wherein a 5HTTLPR Short/Short (S/S) genotype indicates an increased compliance with a noradrenergic and specific serotonergic antidepressant treatment regimen.

10. The method according to claim 8, wherein said noradrenergic and specific serotonergic antidepressant is mirtazapine.

11. The method according to claim 1, further including:
using said prediction to determine an antidepressant treatment regimen for said subject.

12. A method of predicting whether a subject will comply with a serotonin selective reuptake inhibitor (SSRI) treatment regimen, said method comprising:
determining said subject's serotonin transporter gene-linked polymnorphic region (5HTTLPR) genotype; and
using said determined genotype to predict whether said subject will comply with said SSRI treatment regimen.

13. The method according to claim 12, wherein one form of said 5HTTLPR genotype indicates a decreased compliance to a SSRI treatment regimen, compared to other forms of said 5HTTLPR genotype.

14. The method according to claim 12, wherein said subject is a human.

15. The method according to claim 14, wherein said human subject possesses major depressive disorder (MDD).

16. The method according to claim 12, wherein a 5HTTLPR Long/Long (L/L) genotype indicates an increased compliance with a SSRI treatment regimen.

17. The method according to claim 16, wherein said SSRI treatment regimen comprises administration of paroxetine to said subject.

18. The method according to claim 12, further including:
using said prediction to determine an antidepressant treatment regimen for said subject.

19. A method of predicting whether a subject will comply with a noradrenergic and specific serotonergic antidepressant treatment regimen, said method comprising:
determining said subject's serotonin transporter gene-linked polymnorphic region (5HTTLPR) genotype; and
using said determined genotype to predict whether said subject will comply with said noradrenergic and specific serotonergic antidepressant treatment regimen.

20. The method according to claim 19, wherein one form of said 5HTTLPR genotype indicates a decreased compliance to a noradrenergic and specific serotonergic antidepressant treatment regimen, compared to other forms of said 5HTTLPR genotype.

21. The method according to claim 19, wherein said subject is a human.

22. The method according to claim 21, wherein said human subject possesses major depressive disorder (MDD).

23. The method according to claim 19, wherein a 5HTTLPR Short/Short (S/S) genotype indicates an increased compliance with a noradrenergic and specific serotonergic antidepressant treatment regimen.

24. The method according to claim 23, wherein said noradrenergic and specific serotonergic antidepressant treatment regimen comprises administration of mirtazapine to said subject.

25. The method according to claim 19, further including:
using said prediction to determine a noradrenergic and specific serotonergic antidepressant treatment regimen for said subject.

26. A method of predicting whether a subject will discontinue an antidepressant treatment regimen, said method comprising:
determining said subject's serotonin transporter gene-linked polymorphic region (5HTTLPR) genotype;
using said determined genotype to predict whether said subject will discontinue said antidepressant treatment regimen.

27. The method according to claim 26, wherein said antidepressant treatment regimen comprises administration of an antidepressant agent to said subject.

28. The method according to claim 26, wherein said subject is a human.

29. The method according to claim 28, wherein said human subject possesses major depressive disorder (MDD).

30. The method according to claim 27, wherein said antidepressant agent is a serotonin selective reuptake inhibitor (SSRI) so that said antidepressant treatment regimen is a SSRI treatment regimen.

31. The method according to claim 30, wherein a 5HTTLPR Long/Long (L/L) genotype indicates an increased compliance with a SSRI treatment regimen.

32. The method according to claim 30, wherein said SSRI is paroxetine.

33. The method according to claim 27, where said antidepressant agent is a noradrenergic and specific serotonergic antidepressant so that said antidepressant treatment regimen is a noradrenergic and specific serotonergic antidepressant treatment regimen.

34. The method according to claim 33, wherein a 5HTTLPR Short/Short (S/S) genotype indicates an increased compliance with a noradrenergic and specific serotonergic antidepressant treatment regimen.

35. The method according to claim 33, wherein said noradrenergic and specific serotonergic antidepressant is mirtazapine.

36. The method according to claim 26, further including:
using said prediction to determine an antidepressant treatment regimen for said subject.

37. A method for treating a subject for a depression state, comprising:
determining whether a subject is suffering from a depression state;
determining said subject's serotonin transporter gene-linked polymorphic region (5HTTLPR) genotype;
using said determined genotype to select a treatment regimen said subject will comply with; and
administering to said subject said treatment regimen to treat said depression state.

38. A kit comprising
reagents for determining a subject's serotonin transporter gene-linked polymnorphic region (5HTTLPR) genotype; and
instructions for evaluating whether said subject will comply with a serotonin selective reuptake inhibitor (SSRI) treatment regimen.

39. The kit according to claim 38, wherein said kit further included PCR primers.

40. The kit according to claim 38, wherein said kit further included a DNA array.
